(19) United States
(12) Patent Application Publication
(10) Pub. No.: US 2024/0194302 A1
(43) Pub. Date: Jun. 13, 2024

(54) Systems and Methods to Predict Biological Receptor Signal Response

(71) Applicants: David Lafond, Galesburg, MI (US); Christine O'Neil, Jupiter, FL (US)

(72) Inventors: David Lafond, Galesburg, MI (US); Christine O'Neil, Jupiter, FL (US)

(21) Appl. No.: 18/537,138

(22) Filed: Dec. 12, 2023

Related U.S. Application Data
(60) Provisional application No. 63/387,248, filed on Dec. 13, 2022.

(51) Int. Cl. G16C 20/30; G06N 5/022; G16B 15/30; G16C 20/20; G16C 20/70

(52) U.S. Cl. CPC G16C 20/30; G06N 5/022; G16B 15/30; G16C 20/20; G16C 20/70

(57) ABSTRACT

Systems and methods for screening and identifying compounds that bind to biological receptors and stimulate them to produce a signal response. Specifically, the biological receptors include any and all biological receptors that function in flavor and aroma response, as well as any biological receptors utilized for responses tied to various pharmaceuticals. The present invention significantly reduces the time necessary for identifying new compounds in food and pharmaceutical research.

The present invention claims priority to U.S. Prov. Pat. App. No. 63/387,248, titled “A Method to Predict Biological Optimal Signal Response,” filed Dec. 13, 2022, which is incorporated herein by reference in its entirety.

### TECHNICAL FIELD

The present invention relates to systems and methods for screening and identifying compounds that bind to biological receptors and stimulate them to produce a signal response. Specifically, the biological receptors may include any and all biological receptors that function in flavor and aroma response, as well as any biological receptors utilized for responses tied to various pharmaceuticals. The present invention significantly reduces the time necessary for identifying new compounds in food and pharmaceutical research.

### BACKGROUND

Current flavor and pharmaceutical research is focused on chemical structure of compounds and how the structure of the compounds facilitates binding to biological receptors, which, it is believed, causes a signal response from the biological receptors. However, certain chemical compounds generate similar signal responses despite having very different chemical structures. Therefore, it is believed that structure alone cannot be the sole factor in the effects on the receptors to produce a signal. For example, the chemical structure for aspartame is very different from the chemical structure of sugar, yet both chemicals cause a similar signal response as the chemicals bind to receptors: both chemicals produce a sweet perception to a human being.

A factor that does not appear to have been considered is molecular vibration frequency of chemical compounds. Specifically, every molecule vibrates in solution according to atoms bound together (through the bonds) to form the molecule, each electron having their own energy states and orbits. This vibrational frequency may have an impact on the receptors and be at least partly responsible for the chemical binding to receptors and, therefore, the signal responses generated therefrom. In the example above, the molecular vibration frequency of both aspartame and sugar may be partly responsible for the sweet perception.

The chemical vibration frequency may shift from its native state in buffer solution to a different frequency when it is bound to the receptor. These frequencies can be measured using infrared spectroscopy as well as Raman spectroscopy.

Raman spectroscopy is based on Raman scattering, a phenomenon where incident photons gain or lose energy by interacting with vibrating molecules in a sample.

Specifically, the bonds between atoms in a molecule can be approximated as a spring connecting two masses, as noted in Raman Spectroscopy: Techniques and applications in the life sciences, Dustin W. Shipp, Faris Sinjabi, and Ioan Notingher, Advances in Optics and Photonics, 2017. The potential energy U of the “spring” is given by:

U=½kx² (Eq. 1)

where k is the “spring constant,” and x is the displacement of the nuclei from their equilibrium bond position. Applying Schrödinger's equation to this potential gives

(−h⁻²d²ψ)/(2mdx²)+½(kx²ψ)=Eψ (Eq. 2)

where E is the vibration energy and y is the wavefunction of the system. In this equation, m is the reduced mass of the atoms involved in the vibration, given by m=(m₁ m₂)/(m₁+m₂).

Solving Equation 2 reveals that these vibrations are quantized. The vibrational energies are given by:

$$\\ \begin{matrix}{E}_{v} = ( v + 1 / 2 ) \sqrt[h]{k / m} & ( Eq . 3 )\end{matrix}$$

where v is the quantum number of the vibrational mode. This result can be applied to vibrational modes of molecules and shows that the energies are quantized. Indeed, for larger and more complex molecules, the dependence of k and m on the force constants and masses of the atoms is more complicated. Energies of molecular vibrational modes are expressed with units of wavenumbers, or cm⁻¹.

A need, therefore, exists for improved systems and methods for identifying chemical compounds that cause signal responses in biological receptors. Specifically, a need exists for systems and methods for utilizing molecular vibration frequencies as a tool in identifying chemical compounds that cause signal responses in biological receptors. More specifically, a need exists for systems and methods for relating chemical vibration frequencies and chemical structures to identify chemical compounds that cause signal responses in biological receptors.

Moreover, a need exists for systems and methods for identifying chemical compounds that cause signal responses in biological receptors that may be accomplished relatively quickly, thereby reducing the discovery time of new compounds in food and pharmaceutical research, which impacts millions of lives around the world. Specifically, a need exists for systems and methods for identifying chemical compounds that cause signal responses in biological receptors that utilize new data and/or existing measured data. More specifically, a need exists for systems and methods that utilize one or both of the vibrational frequencies of chemical compounds in solution and when bound to biological receptors, respectively, to identify chemical compounds for use in the same.

### SUMMARY OF THE INVENTION

The present invention relates to systems and methods for screening and identifying compounds that bind to biological receptors and stimulate them to produce a signal response. Specifically, the biological receptors may include any and all biological receptors that function in flavor and aroma response, as well as any biological receptors utilized for responses tied to various pharmaceuticals. The present invention significantly reduces the time necessary for identifying new compounds in food and pharmaceutical research.

To this end, in an embodiment of the present invention, a method for identifying one or more chemical compounds that elicits a response by a biological receptor comprising the steps of: obtaining vibration frequency data of at least one chemical compound when the at least one chemical compound is unbound and when the at least one chemical compound is bound to a biological receptor; obtaining chemical structure data of the at least one chemical compound and digitizing the chemical structure data; analyzing the vibration frequency data of the at least one chemical compound when the chemical compound is unbound, when the at least one chemical compound is bound to a biological receptor, and the chemical structure data of the at least one chemical compound; and identifying at least one other chemical compound for binding to the biological receptor based on the vibrational frequency data and the chemical structure data of the at least one chemical compound.

In an embodiment, the vibration frequency data is obtained using Raman spectroscopy.

In an embodiment, the vibration frequency data is obtained when the at least one chemical compound is bound to the biological receptor by measuring a response by the biological receptor.

In an embodiment, the response by the biological receptor is measured by detecting calcium via a calcium sensitive probe.

In an embodiment, the method further comprises the step of: compiling the vibration frequency data of the at least one chemical compound and the digitized chemical structure data in a dataset within a processor comprising a communication module and a memory controlled by the processor, wherein the processor analyzes the vibration frequency data of the at least one chemical compound when the chemical compound is unbound, when the at least one chemical compound is bound to a biological receptor, and the chemical structure data of the at least one chemical compound and identifies at least one other chemical compound for binding to the biological receptor based on the vibrational frequency data and the chemical structure data of the at least one chemical compound

In an embodiment, the method further comprises the steps of: extracting features from the vibration frequency data; and compiling the features extracted from the vibration frequency data into the dataset.

In an embodiment, the method further comprises the step of: using an iterative machine learning tool for building a predictive model, wherein the predictive model is used to identify the at least one other chemical compound for binding to the biological receptor based on the vibrational frequency data and the chemical structure data of the at least one chemical compound.

In an embodiment, the step of identifying at least one other chemical compound for binding to the biological receptor based on the vibrational frequency data and the chemical structure data of the at least one chemical compound is based upon predicting binding affinity of the at least one other chemical compound to the biological receptor.

In an embodiment, the chemical structure data is selected from the group of bond types, functional groups, spatial arrangement data, and combinations thereof.

In an embodiment, the method further comprising the step of: validating the identification of the at least one other chemical compound by evaluating the at least one other compound when bound to the biological receptor.

In an alternate embodiment of the present invention, a system for identifying one or more chemical compounds that elicits a response by a biological receptor is provided. The system comprises: a processor comprising a communication module and a memory controlled by the processor, the memory including instructions that when executed by the processor cause the processor to perform the steps of: analyzing vibration frequency data of at least one chemical compound when the chemical compound is bound to a biological receptor and the chemical structure data of the at least one chemical compound; and identifying at least one other chemical compound for binding to the biological receptor based on the vibrational frequency data and the chemical structure data of the at least one chemical compound.

In an embodiment, the vibration frequency data is obtained using Raman spectroscopy.

In an embodiment, the vibration frequency data is obtained when the at least one chemical compound is bound to the biological receptor by measuring a response by the biological receptor.

In an embodiment, the response by the biological receptor is measured by detecting calcium via a calcium sensitive probe.

In an embodiment, the chemical structure data is digitized by converting the chemical structure data into numerical representations.

In an embodiment, the memory further includes instructions that when executed by the processor cause the processor to perform the steps of: extracting features from the vibration frequency data; and compiling the features extracted from the vibration frequency data into the dataset.

In an embodiment, the memory further includes instructions that when executed by the processor cause the processor to perform the steps of: using an iterative machine learning tool for building a predictive model, wherein the predictive model is used to identify the at least one other chemical compound for binding to the biological receptor based on the vibrational frequency data and the chemical structure data of the at least one chemical compound.

In an embodiment, the step of identifying at least one other chemical compound for binding to the biological receptor based on the vibrational frequency data and the chemical structure data of the at least one chemical compound is based upon predicting binding affinity of the at least one other chemical compound to the biological receptor.

In an embodiment, the chemical structure data is selected from the group of bond types, functional groups, spatial arrangement data, and combinations thereof.

In an embodiment, the memory further includes instructions that when executed by the processor cause the processor to perform the step of: validating the identification of the at least one other chemical compound by evaluating the at least one other compound when bound to the biological receptor.

It is, therefore, an advantage and objective of the present invention to provide improved systems and methods for identifying chemical compounds that cause signal responses in biological receptors.

Specifically, it is an advantage and objective of the present invention to provide systems and methods for utilizing molecular vibration frequencies as a tool in identifying chemical compounds that cause signal responses in biological receptors.

More specifically, it is an advantage and objective of the present invention to provide systems and methods for relating chemical vibration frequencies and chemical structures to identify chemical compounds that cause signal responses in biological receptors.

Moreover, it is an advantage and objective of the present invention to provide systems and methods for identifying chemical compounds that cause signal responses in biological receptors that may be accomplished relatively quickly, thereby reducing the discovery time of new compounds in food and pharmaceutical research, which impacts millions of lives around the world.

Specifically, it is an advantage and objective of the present invention to provide systems and methods for identifying chemical compounds that cause signal responses in biological receptors that utilize new data and/or existing measured data.

More specifically, it is an advantage and objective of the present invention to provide systems and methods that utilize one or both of the vibrational frequencies of chemical compounds in solution and when bound to biological receptors, respectively, to identify chemical compounds for use in the same.

Additional features and advantages of the present invention are described in, and will be apparent from, the detailed description of the presently preferred embodiments and from the drawings.

### DETAILED DESCRIPTION OF THE PRESENTLY PREFERRED EMBODIMENTS

The present invention relates to systems and methods for screening and identifying compounds that bind to biological receptors and stimulate them to produce a signal response. Specifically, the biological receptors may include any and all biological receptors that function in flavor and aroma response, as well as any biological receptors utilized for responses tied to various pharmaceuticals. The present invention significantly reduces the time necessary for identifying new compounds in food and pharmaceutical research.

For the purposes of the present invention, the term “biological receptor” refers to any cell-based receptor in humans or animals that produces a signal response when a chemical compound is bound to the cell-based receptor.

The present invention facilitates the discovery and identification of chemical compounds that may provide signal responses for specific cell-based receptors. Specifically, the present invention utilizes molecular vibrational frequency measurements with chemical structure data of the chemical compounds to identify chemical compounds that may trigger a desired cell-based receptor signal response.

In a first step, molecular vibration frequencies of chemical compounds may be measured using, for example, Raman spectroscopy. Of course, any other method for obtaining molecular vibration frequencies may be utilized and the present invention should not be limited as described herein. The molecular vibration frequencies are measured, preferably, when the chemical compound is in solution and when bound to a biological receptor. Such measurements may provide data relating to, specifically, the change in molecular vibration frequencies when the chemical compound becomes bound to a biological receptor.

For example, a sample chemical compound may be screened for its ability to produce a sweet signal response when bound to the g-coupled protein T1R2 T1R3 receptors. Specifically, the chemical compound may be tested using Raman spectroscopy in solution and when bound to the g-coupled protein T1R2 T1R3 receptors. The response of the g-coupled protein biological receptor may be measured, for example, by measuring for the presence of calcium via a calcium sensitive fluorescent probe.

In a second step, the chemical structure of the chemical compounds are obtained and digitized, using, for example, other measurement techniques or via databases. The chemical structure data may preferably include bond types, functional groups, spatial arrangements and/or any other information concerning chemical structure that may be obtained and utilized.

In a third step, the digitized chemical structure of the chemical compound is combined together with the measured vibrational frequency data and iteratively analyzed to determine patterns of chemical vibration frequency and chemical structure, which may be applied, in a fourth step, to other chemical compounds that may have the same or similar patterns of vibrational frequency and chemical structure.

Iterative analysis, such as analysis that may be performed by an artificially intelligent (AI) system, may significantly increase the recognition of patterns and, therefore, enhance, predictions by analyzing complex datasets and extracting patterns that may not be readily apparent through traditional methods, such as analysis by an individual.

### Examples

In an exemplary embodiment, a first step, as discussed above, may involve the gathering of molecular vibration frequency measurements of a chemical compound of interest. Preferably, this may be done using Raman spectroscopy although it should be noted that data may be provided by previously measured molecular vibration frequency measurements. In addition, it is preferable that the chemical vibrational frequency measurements of the chemical compound be gathered both in solution (i.e., without being bound to a biological receptor) and also while being bound to a biological receptor.

A known receptor binding/response mechanism, such as the aforementioned calcium measurement using a calcium sensitive fluorescent probe imaging analysis, may be utilized to determine whether and how strong a response is to the binding of the chemical compounds with the biological receptors.

The following presents exemplary methodologies according to the present invention. It should be noted that the present invention should not be limited as described herein.

### Exemplary Measurement of Cellular Responses by Calcium-Imaging Analysis

For fluorescence microscopy, in an exemplary embodiment, cells are first seeded onto Lumox multiwell 96-well plates (Starstedt AG & Co.) at approximately 50,000 human receptors per well. After 20-26 hours, the cells are washed with assay buffer and loaded with 5 μm of fura-2-acetoxymethyl ester (fur-2AM; Invitrogen) in assay buffer for 30 minutes at 27° C. The cells are again washed with assay buffer and incubated in 100 μL of assay buffer for up to 15 minutes at room temperature. The cells are stimulated with test compound by adding 100 μL of 2× ligand, i.e., double-strength ligand solution.

The intensities of fura-2 fluorescence emissions resulting from excitations at 340 nm and 380 nm are measured at 510 nm using a computer-controlled Lambda 10-3 filter exchanger (Sutter), a Photometrics CoolSNAP HQ2 charge-coupled device camera, and an inverted IX-71 fluorescence microscope (Olympus). The images are recorded at 4 second intervals and analyzed using Molecular Devices Co. MetaFluor software. Changes in the intra-cellular calcium ion concentrations are estimated from changes in the ratio of the fluorescence intensities at the two excitation wave lengths (i.e., 340 nm and 380 nm).

### Exemplary Measurement of Cellular Responses by Cell-Based Assay

In an exemplary embodiment, multiple data points and dose-response curves are generated in the cell-based assay using a Molecular Devices FlexStation 3. For the multiwell assays, human receptor cells are seeded onto Corning Inc. CellBIND 96-well plates at approximately 80,000 cells per well. Cells are washed with an assay buffer prior to loading with a calcium-indicator dye from the Molecular Devices Co. FLIPR Calcium 4 Assay Kit by dilution with the assay buffer. The cells are incubated for 45 minutes at 27° C., after which measurements are made using the FlexStation 3. Fluorescence changed (i.e., excitation at 485 nm and emission at 525 nm with a cutoff at 515 nm) are monitored at 2 second intervals. A 100 μL aliquot of assay buffer supplemented with 2× ligands is added at 20 s and scanning continued for an additional 100 seconds. The response of each well is determined as delta relative fluorescent units (DRFU), calculated as (maximum fluorescent value)−(minimum fluorescent value). The data are reported as the mean±S.E.M. of the DRFU. Fitting curves for dose-response data and its correlation coefficient values are calculated with Axon Instruments Clampfit 9.2 using Hill's equation.

### Exemplary Measurement of Molecular Vibration Frequency

In an exemplary embodiment, the IR spectrum of solid state test compounds are recorded using a Bruker Fourier Transform IFS 113V spectrometer. 100 scans are accumulated at 2 cm⁻¹ resolution for this compound. The Raman spectrum is recorded directly from the test sample of a T 64000 ISA/Jobin Yvon spectrometer with 641.5 nm laser radiation and at 2 cm⁻¹ resolution. Depending on the test compound, the laser radiation may be changed for optimal measurement results.

In another example, the IR absorptions spectra are monitored using a Nicolet Impact 410 Fourier Transform IR (FTIR) spectrometer, operated under the Omnic IR data software. The spectra of transparent KBr pellets, prepared by grinding 1 mg of crystalline particles of each sample with 100 mg of dry KBr, are recorded in the 400-4000 cm⁻¹ range at spectral resolution of 2 cm⁻¹ with 2000 accumulations.

The Raman spectroscopy are collected with a Jobin/Yvon, LabRam UV HR micro-Raman spectrometer. A diode laser at 784.9 nm (˜10 mW intensity) is used for excitation and focused onto each sample with a 50/0.75 numerical-aperture microscope objective. The scattered light is redirected from the microscope through a sharp edge long wave-pass filter that rejects the excitation laser line and the elastically scattered light, and through a confocal pinhole for increased axial resolution. The scattered light is then focused into a 0.8 m dispersive spectrometer with an air-cooled charge-coupled device (CCD), consisting of 1024×256 pixels. The spectra in the 100 cm⁻¹ to 3000 cm⁻¹ region is monitored while scanning the spectrum across the CCD and moving the grating. Successive recordings of Raman spectra from the samples allows monitoring of their characteristic spectral features. The definition of the measurement parameters and measuring control is done using LabSpec 4.04 software. The Raman spectra contains a varying background overlaying the signal, and this background is removed by fitting it to a high-order polynomial and subsequently subtracting the polynomial from the spectra.

### Exemplary Iterative Machine Learning Computational System for Analysis

In an exemplary embodiment, as noted, the second step involves determining the chemical structure data of the chemical compound of interest, namely the bond types, the functional groups, the spatial arrangement thereof, and any other chemical structure data apparent to one of ordinary skill in the art. In a third step, a dataset may be compiled that includes the biological receptor response data, the vibration frequency data and/or the corresponding chemical structure of the chemical compound of interest. Specifically, computational tools may be utilized to convert chemical structure data into numerical representations that may be recognizable by a computational system. Moreover, vibration frequency data of the chemical compound may further have features extracted therefrom and further compiled into the dataset that includes both the vibrational frequency data and the chemical structure data of the chemical compound of interest.

As noted above, patterns may be recognized in data via an iterative machine learning system may employ algorithms to build predictive models, recognizing patterns of biological response, vibrational frequency data and chemical structure within the compound of interest and other chemical compounds having similar structures and vibrational frequency data. Preferably, an iterative process utilizing machine learning may predict and learn complex relationships between molecular characteristics via molecular vibration frequency data and biological responses.

Vibrational self-consistent field (VCSF) approach may be used to compute the anharmonic vibrational spectra of test compounds. Variants of the VCSF method developed in recent years may be used for this purpose. These VCSF algorithms have been employed in recent years in spectroscopic calculations for a range of biological molecules, and generally yield results in very good accord with experiments. The VCSF variants may thus be realistically applicable to chemical compounds of interest and may be used directly with potentials from electronic structure codes, generated as points on suitable multidimensional grids.

### Exemplary Validation of Predictive Analysis

In an exemplary embodiment, the accuracy of any predictive analysis may be validated using cross-validation techniques, such as utilizing known compounds known to elicit a receptor response. Predictive models may then be fine-tuned via validation checks to enhance performance and generalizability.

The predictive model generated thereby may be utilized by machine learning tools, such as AI systems, to predict receptor binder affinity or signaling response for new molecules. The new molecules may then be tested against experimental data to assess the accuracy and reliability of the predictive model. As such, the predictive model may thus be utilized to help identify previously unknown chemical compounds that may elicit responses in biological receptors, thereby significantly decreasing the time for identifying and testing the compounds. The predictive model may be continuously refined by incorporating new data and updating the model architecture to improve the predictive capabilities thereof.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

I claim:

1. A method for identifying one or more chemical compounds that elicits a response by a biological receptor comprising the steps of:
obtaining vibration frequency data of at least one chemical compound when the at least one chemical compound is unbound and when the at least one chemical compound is bound to a biological receptor;
obtaining chemical structure data of the at least one chemical compound and digitizing the chemical structure data;
analyzing the vibration frequency data of the at least one chemical compound when the chemical compound is unbound, when the at least one chemical compound is bound to a biological receptor, and the chemical structure data of the at least one chemical compound; and
identifying at least one other chemical compound for binding to the biological receptor based on the vibrational frequency data and the chemical structure data of the at least one chemical compound.

2. The method of claim 1 wherein the vibration frequency data is obtained using Raman spectroscopy.

3. The method of claim 1 wherein the vibration frequency data is obtained when the at least one chemical compound is bound to the biological receptor by measuring a response by the biological receptor.

4. The method of claim 3 wherein the response by the biological receptor is measured by detecting calcium via a calcium sensitive probe.

5. The method of claim 1 further comprising the step of:
compiling the vibration frequency data of the at least one chemical compound and the digitized chemical structure data in a dataset within a processor comprising a communication module and a memory controlled by the processor, wherein the processor analyzes the vibration frequency data of the at least one chemical compound when the chemical compound is unbound, when the at least one chemical compound is bound to a biological receptor, and the chemical structure data of the at least one chemical compound and identifies at least one other chemical compound for binding to the biological receptor based on the vibrational frequency data and the chemical structure data of the at least one chemical compound

6. The method of claim 1 further comprising the step of:
extracting features from the vibration frequency data; and
compiling the features extracted from the vibration frequency data into the dataset.

7. The method of claim 1 further comprising the step of:
using an iterative machine learning tool for building a predictive model, wherein the predictive model is used to identify the at least one other chemical compound for binding to the biological receptor based on the vibrational frequency data and the chemical structure data of the at least one chemical compound.

8. The method of claim 1 wherein the step of identifying at least one other chemical compound for binding to the biological receptor based on the vibrational frequency data and the chemical structure data of the at least one chemical compound is based upon predicting binding affinity of the at least one other chemical compound to the biological receptor.

9. The method of claim 1 wherein the chemical structure data is selected from the group of bond types, functional groups, spatial arrangement data, and combinations thereof.

10. The method of claim 1 further comprising the step of:
validating the identification of the at least one other chemical compound by evaluating the at least one other compound when bound to the biological receptor.

11. A system for identifying one or more chemical compounds that elicits a response by a biological receptor comprising:
a processor comprising a communication module and a memory controlled by the processor, the memory including instructions that when executed by the processor cause the processor to perform the steps of:
analyzing vibration frequency data of at least one chemical compound when the chemical compound is bound to a biological receptor and the chemical structure data of the at least one chemical compound; and
identifying at least one other chemical compound for binding to the biological receptor based on the vibrational frequency data and the chemical structure data of the at least one chemical compound.

12. The system of claim 11 wherein the vibration frequency data is obtained using Raman spectroscopy.

13. The system of claim 11 wherein the vibration frequency data is obtained when the at least one chemical compound is bound to the biological receptor by measuring a response by the biological receptor.

14. The system of claim 13 wherein the response by the biological receptor is measured by detecting calcium via a calcium sensitive probe.

15. The system of claim 11 wherein the chemical structure data is digitized by converting the chemical structure data into numerical representations.

16. The system of claim 11 wherein the memory further includes instructions that when executed by the processor cause the processor to perform the steps of:
extracting features from the vibration frequency data; and
compiling the features extracted from the vibration frequency data into the dataset.

17. The system of claim 11 wherein the memory further includes instructions that when executed by the processor cause the processor to perform the steps of:
using an iterative machine learning tool for building a predictive model, wherein the predictive model is used to identify the at least one other chemical compound for binding to the biological receptor based on the vibrational frequency data and the chemical structure data of the at least one chemical compound.

18. The system of claim 11 wherein the step of identifying at least one other chemical compound for binding to the biological receptor based on the vibrational frequency data and the chemical structure data of the at least one chemical compound is based upon predicting binding affinity of the at least one other chemical compound to the biological receptor.

19. The system of claim 11 wherein the chemical structure data is selected from the group of bond types, functional groups, spatial arrangement data, and combinations thereof.

20. The system of claim 11 wherein the memory further includes instructions that when executed by the processor cause the processor to perform the step of:
validating the identification of the at least one other chemical compound by evaluating the at least one other compound when bound to the biological receptor.
